(19) United States
(12) Patent Application Publication
(10) Pub. No.: US 2010/0081936 A1
(43) Pub. Date: Apr. 1, 2010

(54) ULTRASONIC DIAGNOSIS APPARATUS AND ULTRASONIC TRANSMISSION/RECEPTION METHOD

(76) Inventor: Kazuhito NAKATA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/568,110

(22) Filed: Sep. 28, 2009

(30) Foreign Application Priority Data
Sep. 29, 2008 (JP) 2008-251125

(51) Int. Cl. A61B 8/14

(52) U.S. Cl. 600/447; 600/459

(57) ABSTRACT

An ultrasonic diagnostic apparatus includes an ultrasonic probe including a plurality of ultrasonic transducers, a transmission driving signal generating unit which generates transmission driving signals for forming a plurality of transmitting beams in one ultrasonic transmission and supplies the transmission driving signals to the respective ultrasonic transducers, a calculation unit which calculates delay times of the transmission driving signals for forming the transmitting beams for the respective ultrasonic transducers so as to arrange the respective transmitting beams in centers of fundamental receiving parallel/simultaneous beam groups with a plurality of receiving beams being a unit in which a plurality of receiving beams formed based on the reflected waves are arranged three-dimensionally at equal or equiangular intervals, and a control unit which controls the transmission driving signal generating unit based on each of the calculated delay times.

### CROSS-REFERENCE TO RELATED APPLICATIONS

This application is based upon and claims the benefit of priority from prior Japanese Patent Application No. 2008-251125, filed Sep. 29, 2008, the entire contents of which are incorporated herein by reference.

### BACKGROUND OF THE INVENTION

1. Field of the Invention

The present invention relates to an ultrasonic diagnosis apparatus which can increase an image acquisition rate while maintaining image quality and an ultrasonic transmission/reception method used for the ultrasonic diagnosis apparatus.

2. Description of the Related Art

An ultrasonic diagnosis apparatus is a diagnosis apparatus which transmits ultrasonic pulse waves generated from a plurality of ultrasonic transducers included in an ultrasonic probe, receives reflected ultrasonic waves caused by a difference of acoustic impedance between biological tissues with the plurality of ultrasonic transducers, generates and displays an ultrasonic image. This apparatus is used as a useful apparatus for real-time noninvasive observation,

The ultrasonic diagnostic apparatus is inexpensive and free from radiation exposure as compared with other image diagnosis apparatuses such as an X-ray diagnosis apparatus and an X-ray computer tomographic apparatus. Owing to such characteristics, the application range of ultrasonic diagnosis apparatuses is wide. That is, such apparatuses are used for diagnosis of circulatory organs such as the heart, abdominal regions such as the liver and kidneys, peripheral vessels, diagnosis in obstetrics and gynecology, diagnosis for cerebral blood vessels, and the like.

Recently, such an ultrasonic diagnosis apparatus has used a technique of displaying a realtime three-dimensional ultrasonic image (RT3D image) by ultrasonic scanning (three-dimensional volume scanning) of a three-dimensional region using an electronic or mechanical system. In addition, methods used to increase the volume rate of three-dimensional ultrasonic images include a technique of simultaneously receiving (parallelly/simultaneously receiving) a plurality of beams and a technique of combining transmitting beams in a plurality of directions and transmitting the resultant beam.

In the field of ultrasonic diagnosis, there have been demands to increase the acquisition rate (data acquisition rate) of echo signals regardless of whether two-dimensional planar scanning or three-dimensional volume scanning is performed. For example, in order to increase the volume rate of three-dimensional ultrasonic images, the number of beams to be parallelly/simultaneously received is preferably at least four, more preferably, eight to 32.

Conventional ultrasonic diagnosis apparatuses, however, suffer from the following problems.

For example, as a typical example of conventional three-dimensional volume scanning, three-dimensional volume scanning like that shown in FIG. 12 is executed by parallel/simultaneous reception using 4×2=8 beams like that shown in FIG. 10 or parallel/simultaneous reception using 4×4=16 beams like that shown in FIG. 11. In such an arrangement, receiving beams R differ in distance from a transmitting beam center axis T. This leads to ununiform sensitivity among parallelly/simultaneously received beams. In addition, in order to posteriorly correct the sensitivity ununiformity among the receiving beams by signal processing, it is necessary to use a complicated circuit (filter) for subsequent image processing or the like. Furthermore, it is difficult to completely eliminate inter-beam ununiformity.

### BRIEF SUMMARY OF THE INVENTION

The present invention has been made in consideration of the above situation, and has as its object to provide an ultrasonic diagnosis apparatus which can execute ultrasonic transmission that increases the signal acquisition rate while maintaining image quality and to provide an ultrasonic transmission/reception method used for the ultrasonic diagnosis apparatus.

According to an aspect of the present invention, there is provided an ultrasonic diagnosis apparatus which comprises: an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object; a transmission driving signal generating unit which generates transmission driving signals for forming a plurality of transmitting beams in one ultrasonic transmission and supplies the transmission driving signals to the respective ultrasonic transducers; a calculation unit which calculates delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are arranged three-dimensionally at one of equal and equiangular intervals; and a control unit which controls the transmission driving signal generating unit based on each of the calculated delay times.

According to another aspect of the present invention, there is provided an ultrasonic diagnosis apparatus which comprises: an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object; a transmission driving signal generating unit which generates transmission driving signals for forming a plurality of transmitting beams in one ultrasonic transmission and supplies the transmission driving signals to the respective ultrasonic transducers; a calculation unit which calculates delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are concentrically arranged; and a control unit which controls the transmission driving signal generating unit based on each of the calculated delay times.

According to yet another aspect of the present invention, there is provided an ultrasonic transmission/reception method of transmitting and receiving ultrasonic waves by using an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object, which comprises: generating transmission driving signals for forming a plurality of transmitting beams in one ultrasonic transmission and supplying the transmission driving signals to the respective ultrasonic transducers; calculating delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are arranged three-dimensionally at one of equal and equiangular intervals; and controlling the transmission driving signal generation unit based on each of the calculated delay times.

According to yet another aspect of the present invention, there is provided an ultrasonic transmission/reception method of transmitting and receiving ultrasonic waves by using an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object, which comprises: generating transmission driving signals for forming a plurality of transmitting beams in one ultrasonic transmission and supplying the transmission driving signals to the respective ultrasonic transducers; calculating delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are concentrically arranged; and controlling the transmission driving signal generation based on each of the calculated delay times.

### BRIEF DESCRIPTION OF THE SEVERAL VIEWS OF THE DRAWING

FIG. 1 is a block diagram showing the arrangement of an ultrasonic diagnosis apparatus according to an embodiment of the present invention;

FIG. 2 is a block diagram for explaining the arrangement of an ultrasonic transmission unit 21 according to the embodiment of the present invention;

FIG. 3 is a view for explaining the concept of a reception sensitivity uniforming transmission function when parallel/simultaneous reception according to the embodiment of the present of the invention is executed by using 4×2=8 beams;

FIG. 4 is a view for explaining the concept of the reception sensitivity uniforming transmission function when parallel/simultaneous reception according to the embodiment of the present of the invention is executed by using 4×4=16 beams;

FIG. 5 is a view for explaining the three-dimensional volume (ultrasonic) scanning, using a two-dimensional ultrasonic probe, according to the embodiment of the present invention;

FIG. 6 is a view for explaining a modification of the reception sensitivity ununiforming transmission function when parallel/simultaneous reception according to a conventional method of the prior art is executed by using 1×8=8 beams with one-dimensional array probe;

FIG. 7 is a view for explaining a modification of the reception sensitivity uniforming transmission function when parallel/simultaneous reception according to the embodiment of the present invention is executed by using 1×8=8 beams with one-dimensional array probe;

FIG. 8 is a flowchart showing a procedure for reception sensitivity uniforming transmission processing according to the embodiment of the present invention;

FIG. 9 is a view for explaining a modification of the reception sensitivity uniforming transmission function when parallel/simultaneous reception beams according to the embodiment of the present invention are arrayed concentrically;

FIG. 10 is a view for explaining a modification of the reception sensitivity ununiforming transmission function when parallel/simultaneous reception according to a conventional method of the prior art is executed by using 4×2=8 beams;

FIG. 11 is a view for explaining a modification of the reception sensitivity ununiforming transmission function when parallel/simultaneous reception according to a conventional method of the prior art is executed by using 4×4=16 beams; and

FIG. 12 is a view for explaining a modification of the reception sensitivity ununiforming transmission function with the three-dimensional volume (ultrasonic) scanning, using a two-dimensional ultrasonic probe, according to a conventional method of the prior art.

### DETAILED DESCRIPTION OF THE INVENTION

An embodiment of the present invention will be described below with reference to the views of the accompanying drawing. Note that the same reference numerals denote constituent elements having almost the same functions and arrangements, and a repetitive description will be made only when required.

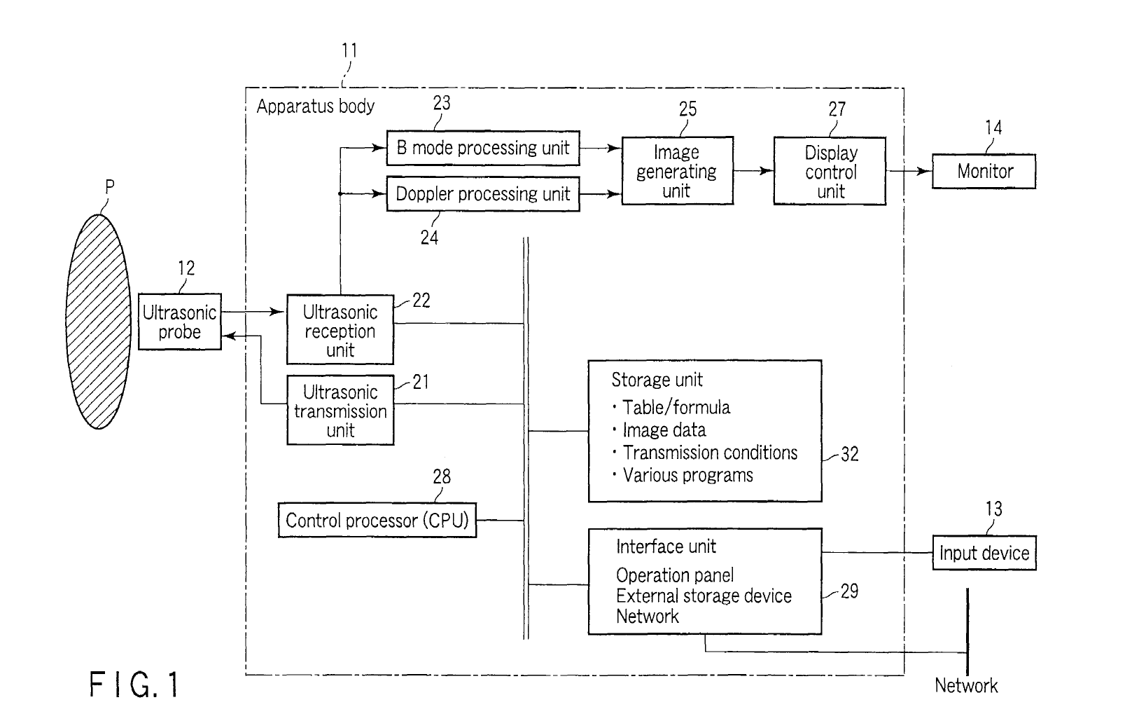

FIG. 1 is a block diagram showing the arrangement of an ultrasonic diagnosis apparatus 10 according to this embodiment.

As shown in FIG. 1, the ultrasonic diagnosis apparatus 10 includes an apparatus body 11, an ultrasonic probe 12, an external input device 13 which is connected to the apparatus body 11 to input various kinds of commands, instructions, and information from an operator to the apparatus body 11, and a monitor 14. The input device 13 includes a trackball, switches/buttons, mouse, and keyboard to, for example, input transmission conditions and set a region of interest (ROI). The apparatus body 11 also includes an ultrasonic transmission unit 21, an ultrasonic reception unit 22, a B mode processing unit 23, a Doppler processing unit 24, an image generating unit 25, a display control unit 27, a control processor (CPU) 28, an interface unit 29, and a storage unit 32.

The ultrasonic probe 12 includes a plurality of ultrasonic transducers serving as acoustoelectric reversible conversion elements such as piezoelectric ceramic elements. The plurality of ultrasonic transducers are juxtaposed (arranged in one-dimension or two-dimension) and provided at the distal end of the probe 12. Each ultrasonic transducer generates an ultrasonic wave at a predetermined timing in accordance with a supplied transmission driving signal (transmission voltage pulse). An ultrasonic wave from each ultrasonic transducer forms a transmitting beam. The transmitting beam is reflected by the discontinuity surface of acoustic impedance in an object. Each ultrasonic transducer receives this reflected wave and generates an echo signal. The ultrasonic reception unit 22 receives these echo signals for each channel.

Note that the ultrasonic probe 12 can be a one-dimensional array probe having a plurality of ultrasonic transducers arrayed along one direction or a two-dimensional array probe having a plurality of ultrasonic transducers arrayed in the form of a two-dimensional matrix.

After an ultrasonic transmitting beam is applied to the object, the ultrasonic reception unit 22 receives echo signals output from the probe 12 for each channel.

The ultrasonic transmission unit 21 is a device (unit) to supply transmission driving signals (transmission voltage pulses) for ultrasonic transmission to the ultrasonic probe 12, and includes a plurality of transmission units 210 for each channel.

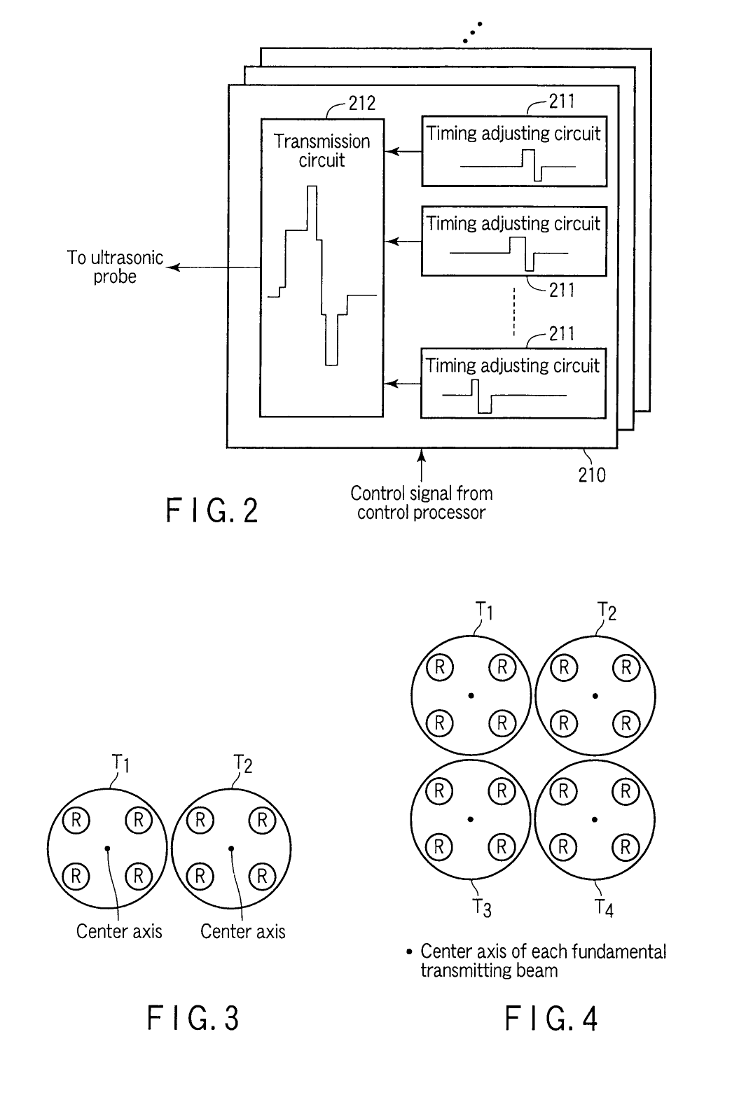

FIG. 2 is a block diagram for explaining the arrangement of the ultrasonic transmission unit 21. As shown in FIG. 2, the ultrasonic transmission unit 21 includes a plurality of transmission units 210 provided for the respective ultrasonic transducers. Each transmission unit 210 includes a plurality of timing adjusting circuits 211 and a transmission circuit 212.

The plurality of timing adjusting circuits 211 of each transmission unit 210 are provided in correspondence with at least the number of beams to be parallelly transmitted. Each timing adjusting circuit 211 generates a transmission voltage pulse control signal S (see FIG. 2) having a delay time and supplied to a corresponding ultrasonic transducer under the control of the control processor 28.

In this case, the delay time to be given to a transmission voltage pulse control signal supplied to each transmission circuit 212 is calculated for each ultrasonic transducer to implement reception sensitivity uniforming transmission (to be described later) (i.e., to focus transmission ultrasonic waves into a beam and have transmission directivity that the position of each fundamental transmitting beam is located equidistant from the respective receiving beams of a fundamental parallel/simultaneous receiving beam group).

The transmission circuit 212 adds/composes control signals S (transmission voltage pulse control signals, control signals for transmission-voltage pulse), which are delay-processed for each transmitting beam by the timing adjusting circuits 211 of each transmission unit 210 for each ultrasonic transducers, on the basis of a rate pulse generated under the control of the control processor 28 (a basic rate signal of ultrasonic transmission/reception).

The ultrasonic reception unit 22 amplifies echo signals received from the ultrasonic probe 12 for each channel, and gives each signal a delay time necessary for the determination of reception directivity. The ultrasonic reception unit 22 then adds the resultant signals. This addition emphasizes reflection components from a direction corresponding to the reception directivity and forms (idealizes) ultrasonic receiving beams (reception directivity).

In particular, in order to acquire parallel/simultaneous receiving beams corresponding to a plurality of directions in response to each transmitting beam, the ultrasonic reception unit 22 concurrently executes delay-and-sum processing a plurality of number of times corresponding to the number of receiving beams by giving a plurality of different reception delays corresponding to the number of beams to the obtained echo signals. Note that the overall directivity (generally called a “scanning line”) of ultrasonic transmission/reception is determined by the reception directivity and transmission directivity. The echo signal after this addition is sent to the B mode processing unit 23 and the Doppler processing unit 24.

Although not shown, the B mode processing unit 23 includes a logarithmic converter and an envelope detection circuit. The logarithmic converter logarithmically converts an echo signal. The envelope detection circuit detects the envelope of the output signal from the logarithmic converter. This detection signal is output as detection data to the image generating unit 25.

The Doppler processing unit 24 extracts a blood flow component by using the analysis result obtained by frequency analysis or a filter, and obtains blood flow information such as an average velocity, variance, and power at many points (sampling points).

The image generating unit 25 executes frame correlation processing and the like by using the detection data constituted by scanning line signal strings input from the B mode processing unit 23, and then generates a B mode image by converting the resultant data into orthogonal coordinate system data based on spatial information. The image generating unit 25 also generates an average velocity image, a variance image, a power image, and their composite image by using the blood flow information input from the Doppler processing unit 24.

The display control unit 27 combines the ultrasonic image received from the image generating unit 25 with predetermined information (e.g., character information, a designated ROI, and the like), and outputs the resultant information to the monitor 14.

The control processor 28 reads out transmission/reception conditions, apparatus control programs, and the like stored in the storage unit 32 based on the mode selection, ROI setting, and various kinds of commands such as transmission start and end commands input from the input device 13 of the user or via a network. The control processor 28 statically or dynamically controls the ultrasonic diagnosis apparatus in accordance with these pieces of information. In addition, the control processor 28 reads out a dedicated program stored in the storage unit 32, and controls the ultrasonic transmission unit 21 and the like in accordance with the program to implement the reception sensitivity uniforming transmission function (to be described later). The control processor 28 also calculates a delay time for each transducer based on transmission conditions including a fundamental transmitting beam count n and a receiving beam count m for each fundamental transmitting beam count in processing (reception sensitivity uniforming transmission processing) conforming to the reception sensitivity uniforming transmission function. The control processor 28 calculates this delay time for each transducer based on, for example, a formula stored in the storage unit 32.

The storage unit 32 records signal data (raw data) received from the B mode processing unit 23 and the Doppler processing unit 24 and image data (still and moving images) received from the image generating unit 25 under the control of the control processor 28. The storage unit 32 also stores control programs for this apparatus, various kinds of data groups such as a diagnosis protocol and transmission/reception conditions, and the dedicated program for implementing the reception sensitivity uniforming transmission function. In addition, the storage unit 32 stores information concerning a predetermined formula for calculating a delay time for each ultrasonic transducer based on a table of correspondence between combinations of fundamental transmitting beam counts n and fundamental receiving beam counts m for the respective fundamental transmitting beams and delay times for the respective ultrasonic transducers and the fundamental transmitting beam count n and the receiving beam count m for each fundamental transmitting beam count.

The monitor 14 displays morphological information in the living body or blood flow information as a still or moving image based on a video signal from the display control unit 27.

### (Reception Sensitivity Uniforming Transmission Function)

The reception sensitivity uniforming transmission function of the ultrasonic diagnosis apparatus 10 will be described next. This function performs transmission control on a plurality of receiving beams which are parallelly/simultaneously received in ultrasonic reception, when, for, example, the receiving beams are arrayed three-dimensionally at equal intervals or equiangular with reference to each of center axis of receiving beams (each of transmission/reception center axis), such that fundamental transmitting beams implementing ultrasonic transmission acoustic field each are positioned in the center of a corresponding fundamental parallel/simultaneous receiving beam group with 2×2 beams being a unit. In this case, a fundamental parallel/simultaneous receiving beam group means a plurality of receiving beams for the center axis position of one transmitting beam. In addition, the center of a fundamental parallel/simultaneous receiving beam group means the central position defined by using the center axis of each receiving beam constituting the fundamental parallel/simultaneous receiving beam group.

FIG. 3 is a view for explaining the concept of the reception sensitivity uniforming transmission function. This function uses two fundamental transmitting beams T1 and T2. In addition, a fundamental parallel/simultaneous receiving beam group corresponding to the fundamental transmitting beam T1 includes 2×2=4 receiving beams R included in the transmitting beam T1. A fundamental parallel/simultaneous receiving beam group corresponding to the fundamental transmitting beam T2 includes 2×2=4 receiving beams R included in the transmitting beam T2. When parallel/simultaneous reception using 4×2=8 beams is to be performed in this manner, this function controls the delay times of transmission driving signals to be supplied to the respective ultrasonic transducers so as to position the center axis of the fundamental transmitting beams T1 and T2 in the centers of the corresponding fundamental parallel/simultaneous receiving beam groups.

FIG. 4 is a view showing another form of this reception sensitivity uniforming transmission. This function uses four fundamental transmitting beams T1, T2, T3, and T4. When parallel/simultaneous reception using 4×4=16 beams is to be performed in this manner, this function controls the delay times of transmission driving signals to be supplied to the respective ultrasonic transducers so as to position the center axis of the fundamental transmitting beams T1, T2, T3, and T4 in the centers of the corresponding fundamental parallel/simultaneous receiving beam groups.

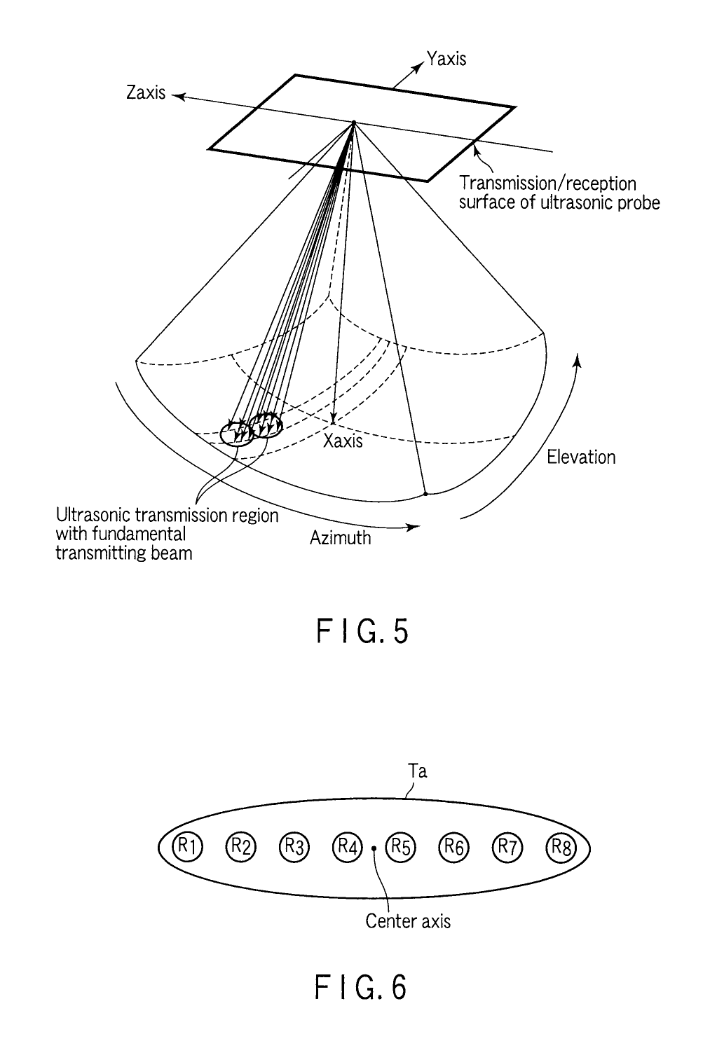

Note that it is possible to generate such a transmitting beam constituted by a plurality of fundamental transmitting beams in the following manner. For example, a transmitting beam constituted by the fundamental transmitting beams T1 and T2 shown in FIG. 3 can be formed as follows. The transmission circuits 212 add a control signal S1 delay-time-processed for implementing the waveform of the fundamental transmitting beam T1 and a control signal S2 delay-time-processed for implementing the waveform of the fundamental transmitting beam T2 for the respective transducers, and supply transmission voltage pulses to the respective transducers in accordance with the control signals obtained by the addition, thereby generating a beam for the simultaneous transmission of the fundamental transmitting beams T1 and T2. When, therefore, three-dimensional volume scanning is to be performed, a three-dimensional region is scanned with the fundamental transmitting beams T1 and T2, as shown in FIG. 5. However, the present invention is not limited to this. For example, when each fundamental transmitting beam is assigned to a group of channels, the fundamental transmitting beams T1 and T2 can be separately transmitted.

FIGS. 3 and 4 exemplify, for a concrete description, the case in which 2×2=4 beams which are equidistant from a fundamental transmitting beam and are parallelly/simultaneously received. However, the present invention is not limited to this. The count m of receiving beams to be parallelly/simultaneously received in correspondence with one fundamental transmitting beam can be any number as long as the receiving beams are arranged equidistant from the central position of the transmitting beam. If, therefore, for example, the count m of receiving beams to be parallelly/simultaneously received for each fundamental transmitting beam is six, a fundamental transmitting beam with receiving beams being arrayed concentrically at equal angular intervals (e.g., at 60° intervals) from the central position of a transmitting beam is used. Even in such a case, ultrasonic transmission for uniform reception sensitivity can be implemented in parallel/simultaneous reception.

In addition, the fundamental transmitting beam count n is properly controlled in accordance with the number of beams to be parallelly/simultaneously received. In the apparatus according to this embodiment, when, for example, parallel/simultaneous reception is to be performed by 4×8=32 beams, fundamental transmitting beam count n=8 is set.

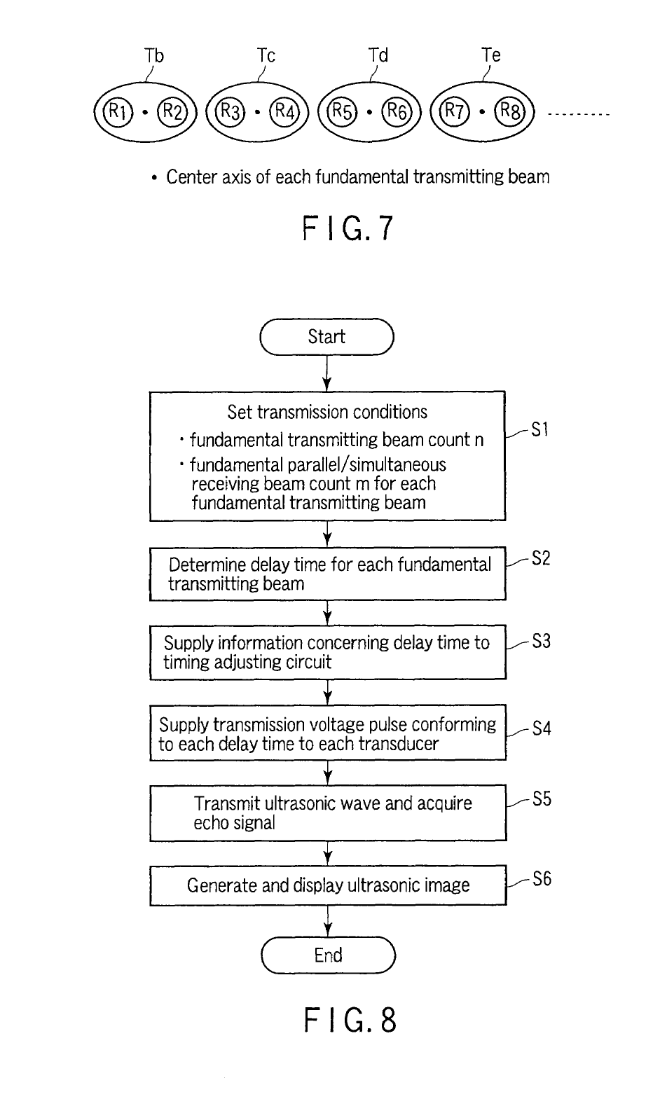

In addition, this reception sensitivity uniforming transmission function is not limited to three-dimensional volume scanning with a two-dimensional array probe, and can also be applied to a one-dimensional array probe. For example, when parallel/simultaneous reception with 1×8=8 beams is to be performed by using a one-dimensional array probe, a transmitting beam Ta whose center axis is located in the middle of receiving beams R1 to R8 arrayed at equal intervals as shown in FIG. 6 is generally used. In this case, conforming to this reception sensitivity uniforming transmission function can form transmitting beams for parallel/simultaneous reception by using fundamental transmitting beams Tb, Tc, Td, and Te each with 1×2=2 beams parallelly/simultaneously received being located equidistant from the center of the corresponding fundamental transmitting beam, as shown in FIG. 7.

### (Operation)

The operation of reception sensitivity uniforming transmission processing by the ultrasonic diagnosis apparatus 10 will be described next.

FIG. 8 is a flowchart showing a procedure for reception sensitivity uniforming transmission processing. As shown in FIG. 8, first of all, transmission conditions including the fundamental transmitting beam count n and the fundamental parallel/simultaneous receiving beam count m for each fundamental transmitting beam are set based on inputs from the input device 13 or information selected from information registered in advance (step S1). Note that, for example, the fundamental transmitting beam count n and the fundamental parallel/simultaneous receiving beam count m for each fundamental transmitting beam are not limited to inputs from the input device 13 or selected information, and preset values can be automatically set as such transmission conditions unless any information for changes is input.

The control processor 28 then determines a delay time for each fundamental transmitting beam corresponding to ultrasonic transducers in accordance with the determined transmission conditions (step S2). The determination of this delay time for each fundamental transmitting beam is executed by using a predetermined formula. The control processor 28 supplies information concerning the determined delay time for each fundamental transmitting beam to each corresponding timing adjusting circuit 211 (step S3).

Each timing adjusting circuit 211 then generates a control signal (a transmission voltage pulse control signal, a control signal of transmission voltage pulse) delay-time-processed based on information concerning the supplied delay time, and outputs the signal to the transmission circuit 212. The transmission circuit 212 adds/composes the control signals S delay-time-processed in each timing adjusting circuit 211 for each transmitting beam, and supplies the transmission driving signal (transmission voltage pulse) to each corresponding transducer on the basis of the added control signals S (step S4).

Each transducer of the ultrasonic probe 12 transmits an ultrasonic beam to the object in accordance with the supplied driving signal (transmission voltage pulse). The respective transmitted beams are sequentially reflected by the discontinuity surface of acoustic impedance of tissue in the object. The respective ultrasonic transducers of the ultrasonic probe 12 acquire the reflected beams as echo signals (step S5).

The ultrasonic reception unit 22 and the B mode processing unit 23 perform predetermined processes for the acquired echo signals, and supply the resultant signals to the image generating unit 25. The image generating unit 25 generates an ultrasonic image based on the supplied echo signals. The monitor 14 displays the generated ultrasonic image in a predetermined form (step S6).

### EFFECTS

According to the above arrangement, the following effects can be obtained.

This ultrasonic diagnosis apparatus performs transmission control on a plurality of receiving beams which are parallelly/simultaneously received in ultrasonic reception, when, for, example, the receiving beams are arrayed three-dimensionally at equal or equiangular intervals with reference to each transmitting beam center axis, such that fundamental transmitting beams implementing ultrasonic transmission each are positioned in the center of a corresponding fundamental parallel/simultaneous receiving beam group with 2×2 beams being a unit. Even if, therefore, the number of receiving beams to be parallelly/simultaneously received is set to about eight to 32, the sensitivity of a plurality of receiving beams to be parallelly/simultaneously received can be made uniform. This makes it possible to increase the signal acquisition rate while maintaining image quality.

In addition, this ultrasonic diagnosis apparatus can arbitrarily control the fundamental transmitting beam count n and the parallel/simultaneous receiving beam count m for each fundamental transmitting beam by predetermined operation and the like through the input device 13. Therefore, by selecting the fundamental transmitting beam count n and the parallel/simultaneous receiving beam count m for each fundamental transmitting beam which are suitable for a desired signal acquisition rate, an ultrasonic image with high image quality can always provided.

Note that the present invention is not limited to the above embodiment, and constituent elements can be variously modified and embodied at the execution stage within the spirit and scope of the invention. For example, the following are concrete modifications.

(1) Each function associated with this embodiment can also be implemented by installing programs for executing the corresponding processing in a computer such as a workstation and mapping them in a memory. In this case, the programs which can cause the computer to execute the corresponding techniques can be distributed by being stored in recording media such as magnetic disks (Floppy® disks, hard disks, and the like), optical disks (CD-ROMs, DVDs, and the like), and semiconductor memories.

(2) The above embodiment has exemplified the case in which a plurality of beams to be parallelly/simultaneously received are arranged equidistant from the center axis of each fundamental transmitting beam at equal intervals. However, the arrangement of beams is not limited to this as long as a plurality of parallel/simultaneous receiving beams are arrayed equidistant from the center axis of each fundamental transmitting beam. Even if, therefore, a plurality of parallel/simultaneous receiving beams are not arrayed equiangularly to the center axis of each fundamental transmitting beam, it is possible to implement the effect of obtaining sensitivity uniformity like the ultrasonic diagnosis apparatus according to this embodiment.

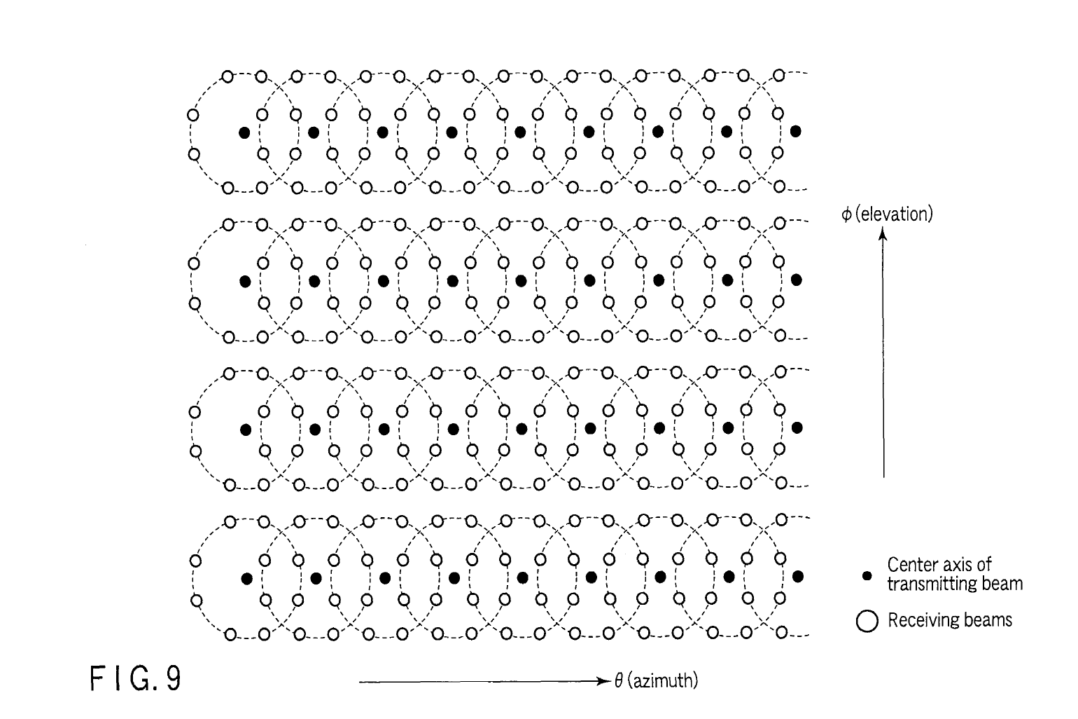

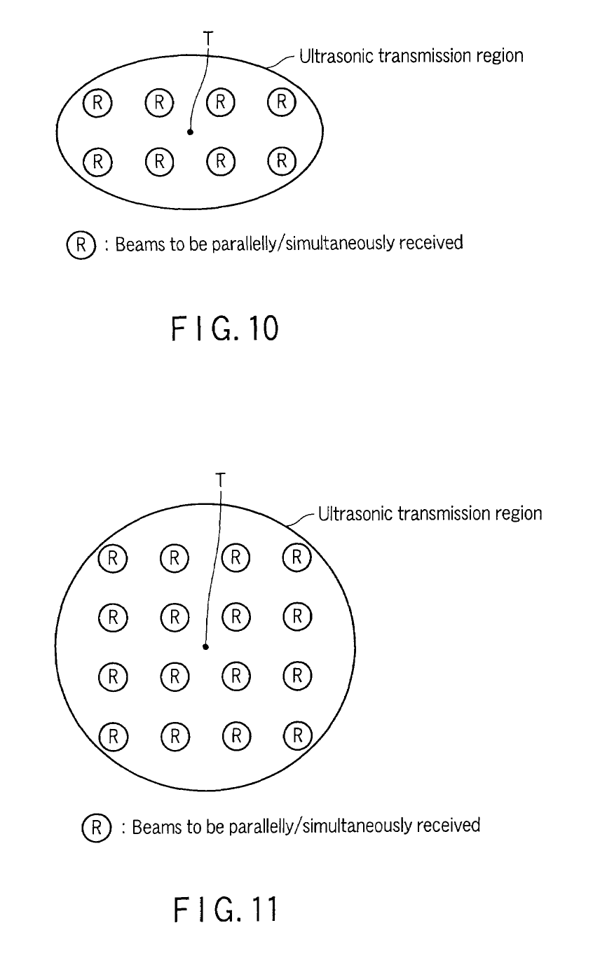

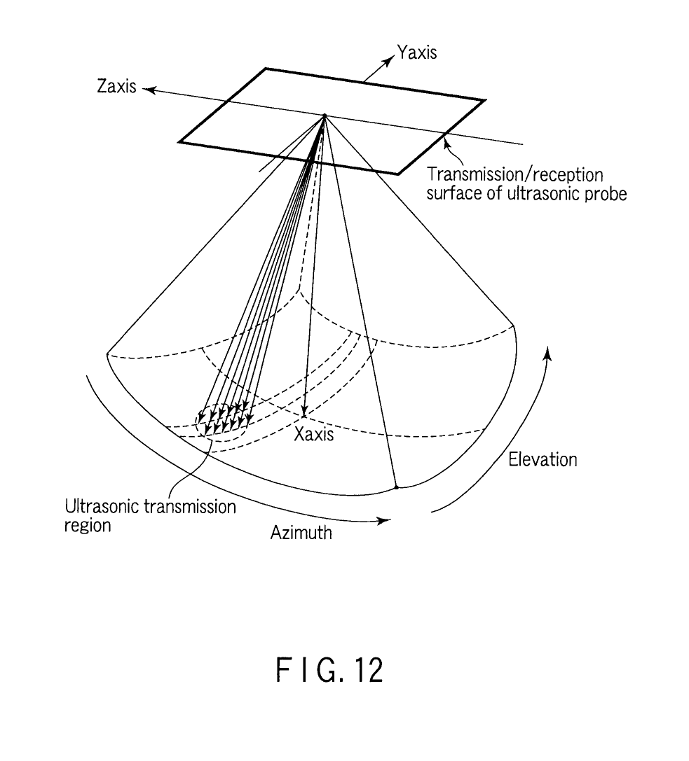

Furthermore, obviously, a fundamental receiving beam group is not limited to a unit of 2×2 beams, and any unit can be used. For example, as shown in FIG. 9, it is possible to use concentrically arrayed eight receiving beams as a fundamental parallel/simultaneous receiving beam group with the center axis of a fundamental transmitting beam being located in the center of the group.

(3) The above embodiment can be configured to actively control a distance r between a plurality of beams to be parallelly/simultaneously received and the center axis of a fundamental transmitting beam or an azimuth θ of a plurality of beams to be parallelly/simultaneously received with respect to the center axis of a fundamental transmitting beam. In such a case, the control processor 28 calculates a delay time for each ultrasonic transducer using the distance r and azimuth θ input from the input device 13 and a predetermined formula.

(4) When transmitting beams arranged in the centers of fundamental parallel/simultaneous receiving beam groups are to be parallelly/simultaneously transmitted in the above embodiment, the transmission order is not limited. For example, in the case shown in FIG. 7, transmitting beams in a plurality of adjacent (spatially continuous) transmission directions may be simultaneously transmitted in combination in such a manner that the transmitting beams Tb and Tc are parallelly/simultaneously transmitted first, and then the transmitting beams Td and Te are parallelly/simultaneously transmitted. Alternatively, transmitting beams in a plurality of transmission directions which are not spatially continuous may be simultaneously transmitted in such a manner that, for example, the transmitting beams Tb and Td are parallelly/simultaneously transmitted first, and then the transmitting beams Tc and Te are parallelly/simultaneously transmitted, or the transmitting beams Tb and Te are parallelly/simultaneously transmitted first, and then the transmitting beam Tc and a transmitting beam Tf are parallelly/simultaneously transmitted.

In addition, various inventions can be formed by proper combinations of a plurality of constituent elements disclosed in the above embodiments. For example, several constituent elements may be omitted from all the constituent elements disclosed in the above embodiments. Furthermore, constituent elements in the different embodiments may be properly combined.

What is claimed is:

1. An ultrasonic diagnosis apparatus comprising:
an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object;
a transmission driving signal generating unit which generates transmission driving signals for forming a plurality of transmitting beams in one ultrasonic transmission and supplies the transmission driving signals to the respective ultrasonic transducers;
a calculation unit which calculates delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are arranged three-dimensionally at one of equal and equiangular intervals; and
a control unit which controls the transmission driving signal generating unit based on each of the calculated delay times.

2. The apparatus according to claim 1, wherein the fundamental parallel/simultaneous receiving beam group includes 2×2 receiving beams as a unit.

3. The apparatus according to claim 1, wherein
the ultrasonic probe comprises a two-dimensional array probe having the plurality of ultrasonic transducers arrayed in the form of a two-dimensional matrix, and
the calculation unit calculates delay times for the respective ultrasonic transducers so as to array the plurality of receiving beams of said each fundamental parallel/simultaneous receiving beam group to be equidistant from a center axis of the transmitting beam.

4. The apparatus according to claim 1, wherein
the ultrasonic probe comprises a one-dimensional array probe having the plurality of ultrasonic transducers arrayed in one direction, and
the calculation unit calculates delay times for the respective ultrasonic transducers so as to array the plurality of receiving beams of said each fundamental parallel/simultaneous receiving beam group at equiangular intervals with reference to a center axis of the transmitting beam.

5. The apparatus according to claim 1, wherein
the ultrasonic probe comprises a one-dimensional array probe having the plurality of ultrasonic transducers arrayed in one direction, and
the calculation unit calculates delay times for the respective ultrasonic transducers so as to array the plurality of receiving beams of said each fundamental parallel/simultaneous receiving beam group to be equidistant from a center axis of the transmitting beam.

6. An ultrasonic diagnosis apparatus comprising:
an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object;
a transmission driving signal generating unit which generates transmission driving signals for forming a plurality of transmitting beams in one ultrasonic transmission and supplies the transmission driving signals to the respective ultrasonic transducers;
a calculation unit which calculates delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are concentrically arranged; and
a control unit which controls the transmission driving signal generating unit based on each of the calculated delay times.

7. The apparatus according to claim 6, wherein the fundamental parallel/simultaneous receiving beam group includes 2×2 receiving beams as a unit.

8. The apparatus according to claim 6, wherein
the ultrasonic probe comprises a two-dimensional array probe having the plurality of ultrasonic transducers arrayed in the form of a two-dimensional matrix, and
the calculation unit calculates delay times for the respective ultrasonic transducers so as to array the plurality of receiving beams of said each fundamental parallel/simultaneous receiving beam group to be equidistant from a center axis of the transmitting beam.

9. The apparatus according to claim 6, wherein
the ultrasonic probe comprises a one-dimensional array probe having the plurality of ultrasonic transducers arrayed in one direction, and
the calculation unit calculates delay times for the respective ultrasonic transducers so as to array the plurality of receiving beams of said each fundamental parallel/simultaneous receiving beam group at equiangular intervals with reference to a center axis of the transmitting beam.

10. The apparatus according to claim 6, wherein
the ultrasonic probe comprises a one-dimensional array probe having the plurality of ultrasonic transducers arrayed in one direction, and
the calculation unit calculates delay times for the respective ultrasonic transducers so as to array the two receiving beams of said each fundamental parallel/simultaneous receiving beam group to be equidistant from a center axis of the transmitting beam.

11. An ultrasonic transmission/reception method of transmitting and receiving ultrasonic waves by using an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object, the method comprising:
generating transmission driving signals for forming a plurality of transmitting beams in one ultrasonic transmission and supplying the transmission driving signals to the respective ultrasonic transducers;
calculating delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are arranged three-dimensionally at one of equal and equiangular intervals; and
controlling the transmission driving signal generation unit based on each of the calculated delay times.

12. An ultrasonic transmission/reception method of transmitting and receiving ultrasonic waves by using an ultrasonic probe including a plurality of ultrasonic transducers which transmit ultrasonic waves to an object based on transmission driving signals supplied to the respective ultrasonic transducers and generate echo signals upon receiving reflected waves of the ultrasonic waves from the object, the method comprising:
generating transmission driving signals for forming a plurality of transmitting beams in one ultrasonic transmission and supplying the transmission driving signals to the respective ultrasonic transducers;
calculating delay times of the transmission driving signals for forming the transmitting beams for the respective ultrasonic transducers so as to arrange the respective transmitting beams in centers of fundamental parallel/simultaneous receiving beam groups with a plurality of receiving beams being a unit in which a plurality of receiving beams formed based on the reflected waves are concentrically arranged; and
controlling the transmission signal generation based on each of the calculated delay times.
